(19) United States
(12) Patent Application Publication
(10) Pub. No.: US 2003/0191046 A1
(43) Pub. Date: Oct. 9, 2003

(54) Cosmetic and perfume products with amber

(76) Inventors: Pawlak Krzysztof, Lodz (PL); Kilian Henryka, Lodz (PL); Macierzynska Ewa, Lodz (PL); Kaczmarska Hanna, Lodz (PL); Wiejacka Jadwiga, Lodz (PL); Kaszczyk Grazyna, Galkow Duzy (PL); Kwiecinska Danuta, Lodz (PL)

Correspondence Address: JASPAN SCHLESINGER HOFFMAN LLP, 300 Garden City Plaza, Garden City, NY, 11530, US

(21) Appl. No.: 10/119,109

(22) Filed: Apr. 9, 2002

(51) Int. Cl. A61K 7/46

(52) U.S. Cl. 512/1; 512/5; 426/76; 426/4

(57) ABSTRACT

Cosmetic and perfume products with amber contain modified amber in the quantity 0.001%-100% by weight.

In order to be used for cosmetics production, amber is primarily exposed to the action of physical or chemical agents or mechanical energy.

An additional cosmetic effect is achieved by adding energizing or active substances as sea alga, yeast extracts, enzymes, liposomes, vitamins and others to modified amber.

The subject of the invention are cosmetic and perfume products with amber in the form of creams, pastes, lotions, cleansers, balms, tonics, fluids, gels, shampoos, hair sprays, conditioners, masks, powders, enamels, enamel removers, lipsticks, foams, cosmetic liquids, cosmetics oils, soaps, peelings, serum, containing modified amber.

Amber is a conifer resin found in nature in the form of lumps or dripstones and it has features of organic origin resins and minerals; it is amorphous, transparent or slightly dull of yellow or brown colour.

In its original form in normal conditions of pressure and temperature, amber is insoluble in water, it is soluble to small degree only in organic solvents as ethyl alcohol, petroleum benzine and benzene.

Up to now amber has been applied mainly as a precious stone and a decoration element in jewellery and artistic products, rarely in pharmaceutical industry in the form of alcohol tincture or a stone in the natural, not ground form in rheumatic diseases.

In its original form amber cannot be used in cosmetics production.

Unexpectedly it was found out that while exposing amber to the action of physical factors as micronization, chemical factors as solving in solvents, alcohols, acids, oils or physical and chemical factors, it possible to modify the original form of amber in such a way that it can be converted for instance into solution or dispersion so that it can be used in cosmetics production.

Amber treated in this way should not lose its natural energizing properties.

It was found out that due to selection of appropriate physical and/or chemical agents, it is possible to preserve energizing properties of amber and at the same time it is possible to convert it into a form which can used in cosmetics production.

Modified amber for cosmetics production can be obtained exposing it to micronization or the action of heat or mechanical energy, microwaves or ultrasounds, or by the action of chemical organic or inorganic and other compounds.

The type of energy, the method it is carried to amber or the intensity of the process decide whether the amber exposed to such treatment preserves its energizing properties or whether loosing them, it gains new ones so that it can be used for instance as a disinfectant.

The agents used for amber modification in order to convert it in a form usable in cosmetics production are: monohydric and polyhydric alcohols, polymerized ethylene oxide and/or its adducts, polar and non-polar solvents or their mixtures, lypophylic compounds (oils), surface-active agents, volatile and non-volatile silicones.

Amber modified for cosmetic purposes can be closed in an envelope in the form of microcapsules and it can also be exposed to treatment by dry distillation.

The content of modified amber in the products according to the invention is 0.001%-100% by weight.

An additional energizing and/or therapeutic effect is achieved by adding energizing substances or other active substances to modified amber.

These substances can be sea origin substances obtained among others from sea alga, plankton, shellfish, yeast extracts, enzymes, vitamins, plant extracts, liposomes and others.

The method of the amber modification process and the raw material being used decide about its application for the defined cosmetic type. So, for instance amber ethanol extract can be used for production of shampoos, hair rinses, perfumes, While lipophylic extracts (oils) can be applied for instance for production of creams and lotions.

To obtain peeling it is possible to use micronized amber and/or amber extracts. An additional cosmetic and/or therapeutic effect can be achieved by adding energizing or active substances.

The cosmetic products according to the invention show energizing or antistress or therapeutic action, which is proved by intensification of skin protective functions as:

improvement of condition of skin which is properly tight, silky smooth and nice in touch,

healthy and visibly younger and fresh skin appearance of delicate gloss,

stimulation of hair growth and improvement of its condition; hair becomes fluffy and glossy, it regains its vitality and health,

nail plates strengthening,

improvement of head skin condition, hair bulbs strengthening, hair losing preventing.

The clue of the invention is presented by the following examples:

### EXAMPLE 1

| | Cosmetic cream | (% by weight) |
|---|---|---|
| 1. | Surface-active substance | 5.0 |
| 2. | Alcohol | 4.0 |
| 3. | Fatty substance | 3.5 |
| 4. | Moisturizing substance | 4.0 |
| 5. | Nourishing substance | 5.0 |
| 6. | Emollients | 4.0 |
| 7. | Modified amber | 7.0 |
| 8. | Energizing (active) substances | 3.0 |
| 9. | Viscosity regulators | 2.0 |
| 10. | Protective substances | 6.0 |
| 11. | Fragrance compound | 0.2 |
| 12. | Preserving agents | 1.0 |
| 13. | Water | up to 100.0 |

### EXAMPLE 2

| | Peeling | (% by weight) |
|---|---|---|
| 1. | Surface-active substance | 5.0 |
| 2. | Alcohol | 5.0 |
| 3. | Fatty substance | 4.0 |
| 4. | Moisturizing substance | 4.0 |
| 5. | Emollients | 3.0 |
| 6. | Modified amber (extract) | 3.0 |
| 7. | Micronized amber | 20.0 |
| 8. | Viscosity regulators | 2.0 |
| 9. | Soothing substances | 5.0 |
| 10. | Fragrance compound | 0.2 |
| 11. | Preserving agents | 1.0 |
| 12. | Water | up to 100.00 |

### EXAMPLE 3

| | Shampoo with amber | (% by weight) |
|---|---|---|
| 1. | Surface-active substances | 18.0 |
| 2. | Modified amber | 2.5 |
| 3. | Energizing substance | 3.0 |
| 4. | Conditioning substance | 2.0 |
| 5. | Substance giving pearly appearance | 2.0 |
| 6. | Enriching substances | 1.0 |
| 7. | Thickening substances | 3.0 |
| 8. | Water | up to 100.0 |

### EXAMPLE 4

| | Hair spray | (% by weight) |
|---|---|---|
| 1. | Resin | 11.8 |
| 2. | Modified amber with energizing substance | 1.0 |
| 3. | Silicone derivative | 0.2 |
| 4. | Neutralizing agent | 0.4 |
| 5. | Fragrance compound | 0.2 |
| 6. | UV filter | 0.3 |
| 7. | Alcohol | up to 100.0 |

### EXAMPLE 5

| | Cosmetic serum | (% by weight) |
|---|---|---|
| 1. | Modified amber | 80.0 |
| 2. | Energizing substance | 18.5 |
| 3. | Viscosity regulators | 1.0 |
| 4. | Preserving agents | 0.3 |
| 5. | Fragrance compound | 0.2 |

### EXAMPLE 6

| | Make-up | (% by weight) |
|---|---|---|
| 1. | Surface-active substance | 5.0 |
| 2. | Alcohols | 4.0 |
| 3. | Fatty substances | 5.0 |
| 4. | Moisturizing substances | 3.0 |
| 5. | Nourishing substances | 2.0 |
| 6. | Emollients | 2.0 |
| 7. | Modified amber | 5.0 |
| 8. | Energizing (enzymatic) substances | 3.0 |
| 9. | Viscosity regulators | 2.0 |
| 10. | Pigments | 10.0 |
| 11. | Fragrance compound | 0.3 |
| 12. | Preserving agents | 10.0 |
| 13. | Water | up to 100.0 |

### EXAMPLE 7

| | Deodorant | (% by weight) |
|---|---|---|
| 1. | Deodoring agent | 5.0 |
| 2. | Modified amber with energizing substance | 3.0 |
| 3. | Enveloping substances | 0.6 |
| 4. | Fragrance compound | 2.0 |
| 5. | Alcohol | up to 100.0 |

1. Cosmetic and perfume products with amber characterized in that they contain modified amber in the quantity 0.001%-100% by weight.

2. Cosmetic and perfume products with amber characterized in that they contain modified amber, energizing substance and/or active substance in the quantity 0.001%-100% by weight.

3. Cosmetic and perfume products according to claim 1 and 2 characterized in that modified amber is in the form alcohol extract of amber.

4. Cosmetic and perfume products according to claim 1 and 2 characterized in that modified amber is in the form extract of amber in polymerized ethylene oxide and/or its adducts.

5. Cosmetic and perfume products according to claim 1 and 2 characterized in that modified amber is in the form extract in polar and/or non-polar solvent.

6. Cosmetic and perfume products according to claim 1 and 2 characterized in that modified amber is in the form lipophylic (oil) extract.

7. Cosmetic and perfume products according to claim 1 and 2 characterized in that modified amber is in the form dispersion in surface-active agents.

8. Cosmetic and perfume products according to claim 1 and 2 characterized in that modified amber is in the form of dispersion in volatile or non-volatile silicone.

9. Cosmetic and perfume products according to claim 1 and 2 characterized in that modified amber is in the form of microcapsules.

10. Cosmetic and perfume products according to claim 2 characterized in that the content of energizing and/or active substance is 0.001% -100% by weight.

11. Cosmetic and perfume products according to claim 2 characterized in that energizing and/or active substance are sea origin raw materials.

12. Cosmetic and perfume products according to claim 2 characterized in that energizing and/or active substance is yeast extract.

13. Cosmetic and perfume products according to claim 2 characterized in that energizing and/or active substance are enzymes.

14. Cosmetic and perfume products according to claim 2 characterized in that energizing and/or active substance are vitamins.

15. Cosmetic and perfume products according to claim 2 characterized in that energizing and/or active substance are vegetable extracts.

16. Cosmetic and perfume products according to claim 2 characterized in that energizing and/or active substance are liposomes.
